SEQUENCE LISTING (8 sequences)

SEQ ID NO: 1 - protein, 240 aa, Escherichia coli CobB
KPRVLVLTGAGISAESGIRTFRAADGLWEEHRVEDVATPEGFDRDPELVQAFYNARRRQLQQPEIQPNAAHLALAKLQDALGDRFLLVTQNIDNLHERAGNTNVIHMHGELLKVRCSQSGQVLDWTGDVTPEDKCHCCQFPAPLRPHVVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 2 - protein, 240 aa, Artificial Sequence
  Dealocase-1
KPRVLVLTGAGISAESGIRTFRAADGLWEEHRVEDVSTPEGFDRDPELVQAFWNAWRRQLQQPEIQPNAAHLALAKLQDALGDRFLLVTQNVDNLHERAGNTNVIHMHGELLKVRCSQSGQVLDWTGDVTPEDKCHCCQFPAPLRPHVVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 3 - protein, 240 aa, Artificial Sequence
  Dealocase-2
KPRVLVLTGAGISAESGIRTFRAADGLWEEHRVEDVGTPEGFDRDPELVQAFYNAGRRQLQQPEIQPNAAHLALAKLQDALGDRFLLVTQNVDNLHERAGNTNVIHMHGELLKVRCSQSGQVLDWTGDVTPEDKCHCCQFPAPLRPHVVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 4 - protein, 240 aa, Artificial Sequence
  Dealocase-3
KPRVLVLTGAGISAESGIRTFRAADGLWEEHRVEDVATPEGFDRDPELVQAFYNARRRQLQQPEIQPNAAHLALAKLQDALGDRFLLVTQNSDNLHERAGNTNVIHMHGELLKVRCSQSGQVLDWTGDVTPEDKCHCCQFPAPLRPHLVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 5 - protein, 240 aa, Artificial Sequence
  Debocylase-1
KPRVLVLTGAGISAESGIRTFRAADGLWEEHRVEDVATPEGFDRDPELVQAFYNARRRQLQQPEIQPNAAHLALAKLQDALGDRFLLVTQNVDNLHERAGNTNVIHMHGELLKVRCSQSGQVLDWTGDVTPEDKCHCCQFPAPLRPHLVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 6 - protein, 240 aa, Artificial Sequence
  Decrotonylase-1
KPRVLVLTGAGISAESGIRTFRAADGLWEEHRVEDVRTPEGFDRDPELVQAFGNATRRQLQQPEIQPNAAHLALAKLQDALGDRFLLVTQNRDNLHERAGNTNVIHMHGELLKVRCSQSGQVLDWTGDVTPEDKCHCCQFPAPLRPHLVWFGEMPLGMDEIYMALSMADIFIAIGTSGHVYPAAGFVHEAKLHGAHTVELNLEPSQVGNEFAEKYYGPASQVVPEFVEKLLKGLKAGSIA

SEQ ID NO: 7 - protein, 550 aa, Photinus pyralis
  firefly luciferase
  SITE 529: chemically inactivated
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 8 - DNA, 1671 bp, Photinus pyralis
  firefly luciferase
atggaggacgcgaaaaacatcaaaaaaggtccggcaccgttttatccgctggaagatggtacagccggtgaacagctgcataaagcaatgaaacgttatgcactggttccgggtacaattgcatttaccgatgcacatattgaagtggatattacctatgccgagtattttgaaatgagcgttcgtctggccgaagccatgaaacgctacggtctgaataccaatcatcgtattgttgtgtgtagcgaaaatagcctgcaatttttcatgccggttctgggtgcactgtttattggtgttgcagttgcaccggcaaatgatatctataatgaacgtgaactgctgaacagcatgggtattagccagccgaccgttgtttttgttagcaaaaaaggcctgcaaaagattctgaacgtgcagaaaaaactgccgatcatccagaaaatcatcatcatggatagcaaaaccgattatcagggtttccagagcatgtatacctttgttaccagccatctgcctccgggttttaacgaatatgattttgttccggaaagcttcgatcgcgataaaaccattgcactgattatgaatagcagcggtagcaccggtctgccgaaaggtgttgcactgccgcatcgtaccgcatgtgttcgttttagccatgcacgtgatccgatttttggcaatcagattattccggataccgcaattctgagcgttgttccgtttcatcatggttttggtatgtttaccacactgggttatctgatttgtggttttcgtgttgttctgatgtatcgctttgaagaagaactgtttctgcgtagtctgcaagattacaaaattcagagcgcactgctggttccgacactgtttagcttttttgccaaaagcaccctgatcgataaatatgatctgagcaacctgcatgaaattgcaagcggtggtgcaccgctgagcaaagaagttggcgaagcagttgccaaacgttttcatctgcctggtattcgtcaaggttatggtctgaccgaaaccaccagtgccattctgattacaccggaaggtgatgataaaccgggtgcagttggtaaagttgtgccgttttttgaagccaaagttgttgatctggataccggtaaaaccctgggtgttaatcagcgtggtgaactgtgtgttcgtggtccgatgattatgagcggttatgttaataatccggaagcaaccaatgcgctgattgataaagatggttggctgcatagcggtgatattgcatattgggatgaagatgaacacttctttattgtggatcgtctgaaaagcctgatcaaatacaaaggttatcaggtggcaccggcagaactggaaagcattctgctgcaacatccgaacatttttgatgcgggtgttgcgggtctgccggatgatgatgcaggcgaactgcctgccgcagttgttgtgctggaacatggcaaaacaatgaccgaaaaagaaatcgttgattatgtggcaagccaggttaccaccgcaaagaaactgcgtggtggtgttgtgtttgttgatgaagttccgaaaggcctgaccggttagctggatgcacgcaaaattcgtgaaattctgatcaaagcgaagaaaggtggtaaatccaagttgcaccatcatcaccaccattaa

(19) United States
(12) Patent Application Publication
(10) Pub. No.: US 2020/0240995 A1
(43) Pub. Date: Jul. 30, 2020

(54) KDAC VARIANTS AND USES THEREOF

(71) Applicant: Max-Planck-Gesellschaft Zur Forderung Der Wissenschaften E.V., Munich (DE)

(72) Inventors: Heinz Neumann, Dortmund (DE); Martin Spinck, Dortmund (DE)

(21) Appl. No.: 16/647,852

(22) PCT Filed: Sep. 21, 2018
(86) PCT No.: PCT/EP2018/075672

§ 371 Date: Mar. 16, 2020

(30) Foreign Application Priority Data
Sep. 22, 2017 (EP) 17192670.2
Apr. 18, 2018 (EP) 18168001.8

(51) Int. Cl. G01N 33/68; C12N 9/02; C12N 9/80; C12Q 1/66

(52) U.S. Cl. CPC G01N 33/6842; C12N 9/0069; C12N 9/80; C12Q 1/66; G01N 2500/02; C12Y 113/12007; C12Y 305/01098; G01N 2440/10; G01N 33/6812

(57) ABSTRACT

The invention provides a method of selecting a mutant polypeptide having lysine demodification, in particular lysine deacylation, activity, wherein the method comprises the following steps (a) incubating a mutant polypeptide having an amino acid sequence with at least 80% sequence identity to SEQ ID NO: 1 with a peptide or polypeptide comprising an inactivated essential lysine residue; and (b) determining the activity of the mutant polypeptide to activate the peptide or polypeptide comprising the inactivated essential lysine residue, wherein the mutant polypeptide and the peptide or polypeptide comprising an inactivated essential lysine residue are incubated in a biological cell. The invention furthermore relates to an acylated luciferase, particularly Firefly luciferase, and uses thereof. The present invention furthermore relates to a mutant polypeptide comprising an amino acid sequence having at least 98% sequence homology with SEQ ID NOs: 2, 3, 4, 5 or 6 and having lysine demodification, in particular lysine deacylation, activity, wherein the mutant polypeptide is not identical to SEQ ID NO: 1. The invention also relates to the mutant polypeptide of the invention and a peptide or polypeptide comprising an inactivated essential lysine residue for use in treating cancer.

### BACKGROUND

The invention provides a method of selecting a mutant polypeptide having lysine demodification, in particular lysine deacylation, activity, wherein the method comprises the following steps (a) incubating a mutant polypeptide having an amino acid sequence with at least 80% sequence identity to SEQ ID NO: 1 with a peptide or polypeptide comprising an inactivated essential lysine residue; and (b) determining the activity of the mutant polypeptide to activate the peptide or polypeptide comprising the inactivated essential lysine residue, wherein the mutant polypeptide and the peptide or polypeptide comprising an inactivated essential lysine residue are incubated in a biological cell. The invention furthermore relates to an acylated luciferase, particularly Firefly luciferase, and uses thereof. The present invention furthermore relates to a mutant polypeptide comprising an amino acid sequence having at least 98% sequence homology with SEQ ID NOs: 2, 3, 4, 5 or 6 and having lysine demodification, in particular lysine deacylation, activity, wherein the mutant polypeptide is not identical to SEQ ID NO: 1. The invention also relates to the mutant polypeptide of the invention and a peptide or polypeptide comprising an inactivated essential lysine residue for use in treating cancer.

Lysine Deacetylases (KDACs) are a prominent class of enzymes featuring roles in almost all physiological processes and many diseases including cancer and aging. These enzymes reverse various types of lysine acylations thereby controlling, e.g., enzyme activities, protein localization and chromatin structure. Acetylation of the NW-amino group of lysine residues was initially discovered fifty years ago on histone proteins. The past two decades revealed a large variety of functional roles of this modification in almost every physiological process. The spectrum of acylations found on lysine side chains is not restricted to acetylation but broad, ranging from short acyl chains to fatty acids and charged functional groups. All these modifications are reversed by a comparably small set of lysine deacetylases (KDACs), which are categorized in four enzyme families. The related class 1, 2 and 4 enzymes are structurally and mechanistically distinct from class 3 KDACs. The formers contain a zinc ion in the active site to orient a water molecule and polarize the substrate, while the latter use NAD+ as a co-substrate to cleave the amide bond. KDACs feature prominently in many physiological processes. Initially discovered on histones, they are well-known as repressors of transcription because removal of the acyl groups enhances histone-DNA contacts and hence leads to chromatin compaction. The discovery of thousands of acylation sites in different organisms from all kingdoms of life gives us an idea of the importance of this modification for the regulation of cellular processes. Defects in these enzymes are connected to a variety of diseases such as diabetes, cancer and aging. Exactly how KDAC misregulation contributes to disease etiology is often difficult to trace because of the limited specificity of the enzymes for particular protein substrates and types of acylation. Genetic ablation of KDACs causes pleiotropic effects mediated by altered gene expression levels. KDAC inhibitors are valuable tools in functional studies and active leads in pharmaceutical design. Unfortunately, their selectivity for particular KDACs is limited, making the interpretation of results more difficult and restricting clinical use.

KDAC variants selective for particular types of lysine modifications would be highly useful. Moreover, there is current need in the art for improved cancer therapies, which cause less severe side-effects and which are highly selective in terms of site of action and time.

Xuan et al., J. Am. Chem. Soc. 139 (2017) 12350-12353, report a genetically encoded fluorescent probe (EGFP-K85AcK) that responds to deacetylases in living cells, which is based on the acetylation of a lysyl residue in EGFP that is essential for chromophore maturation, since correct folding of EGFP, which is required for its fluorescence activity, is prevented by lysine acetylation. Thus, EGFP-K85AcK cannot adopt the native conformation and remains in the unfolded state, so that the acetylated lysine residue is expected to be solvent-exposed and readily accessible for polypeptides with deacetylating activity. While the approach taken by Xuan et al. has been used in an intracellular assay for determining deacetylation activity of deacetylases, it cannot be used as a selection method.

The technical problem underlying the present invention is thus the provision of novel methods for the identification of KDAC variants with improved activity towards the removal of lysine modifications, novel tools for the use in such methods, novel KDAC variants with improved activity towards the removal of lysine modifications and uses thereof.

### SUMMARY OF THE INVENTION

The technical problem is solved by the embodiments as defined in the claims.

In a first aspect, the present invention relates to a method of selecting a polypeptide having lysine demodification, in particular lysine deacylation, activity from a collection of polypeptides, wherein the method comprises the following steps:

(a) incubating said polypeptide with a peptide or polypeptide comprising an essential lysine residue inactivated by a modification, in particular an acylation, of said essential lysine residue; and

(b) selecting said polypeptide based on the ability of said polypeptide to activate said peptide or polypeptide comprising the inactivated essential lysine residue, wherein said polypeptide and said peptide or polypeptide comprising an inactivated essential lysine residue are incubated in a biological cell.

In a second aspect, the present invention relates to method of screening a diverse collection of polypeptides for a polypeptide having lysine demodification, in particular lysine deacylation, activity, wherein the method comprises the following steps:

(a) incubating said diverse collection of polypeptides with a luciferase comprising an inactivated residue K529, wherein said residue is inactivated by a modification, in particular an acylation; and

(b) selecting said polypeptide based on the ability of said polypeptide to activate said luciferase, wherein said diverse collection and said luciferase are incubated in a diverse collection of biological cells; particularly wherein said luciferase is Firefly luciferase according to SEQ ID NO: 7.

In a third aspect, the present invention relates to a method of screening or selecting a KDAC inhibitor from a diverse collection of putative KDAC inhibitors, wherein the method comprises the following steps:

(a) incubating a polypeptide having a lysine demodification, in particular a lysine deacylation, activity with a member of said diverse collection;

(b) adding a peptide or polypeptide comprising an essential lysine residue inactivated by a modification, in particular an acylation, of said essential lysine residue; and

(c) identifying a KDAC inhibitor by the ability to inhibit the demodification, in particular the deacetylation, activity of said polypeptide, wherein the KDAC inhibiting activity of said KDAC inhibitor is reciprocal to the activity of said polypeptide to activate the peptide or polypeptide comprising the inactivated essential lysine residue; in particular, wherein the method is performed in a biological cell.

In a fourth aspect, the present invention relates to a luciferase, particularly a luciferase comprising an amino acid sequence having at least 90% sequence homology to SEQ ID NO: 7, wherein the polypeptide comprises an inactivated lysine residue at a position corresponding to position 529 of SEQ ID NO: 7; particularly wherein the polypeptide comprises the sequence according to SEQ ID NO: 7.

In a fifth aspect, the present invention relates to a nucleic acid encoding the polypeptide of the present invention, wherein the codon encoding the essential lysine residue is replaced by an amber stop codon.

In a sixth aspect, the present invention relates to a mutant polypeptide comprising an amino acid sequence having at least 98, preferably 99% sequence homology with SEQ ID NOs: 2, 3, 4, 5 or 6 and having lysine demodification, in particular lysine deacylation, activity, wherein the mutant polypeptide is not identical to SEQ ID NO: 1.

### DETAILED DESCRIPTION OF THE INVENTION

In a first aspect, the present invention relates to a method of selecting a polypeptide having lysine demodification, in particular lysine deacylation, activity from a collection of polypeptides, wherein the method comprises the following steps:

(a) incubating said polypeptide with a peptide or polypeptide comprising an essential lysine residue inactivated by a modification, in particular an acylation, of said essential lysine residue; and

(b) selecting said polypeptide based on the ability of said polypeptide to activate said peptide or polypeptide comprising the inactivated essential lysine residue,

wherein said polypeptide and said peptide or polypeptide comprising an inactivated essential lysine residue are incubated in a biological cell.

In a particular embodiment the method of said first aspect further comprises the following counter-selection steps:

(c) incubating a polypeptide selected in step (b) with a peptide or polypeptide comprising an essential lysine residue differentially inactivated by a modification different from the modification used in step (a); and

(d) selecting said polypeptide based on the inability of said polypeptide to activate said peptide or polypeptide comprising said differentially inactivated essential lysine residue.

In a second aspect, the present invention relates to method of screening a diverse collection of polypeptides for a polypeptide having lysine demodification, in particular lysine deacylation, activity, wherein the method comprises the following steps:

(a) incubating said diverse collection of polypeptides with a luciferase comprising an inactivated residue K529, wherein said residue is inactivated by a modification, in particular an acylation; and

(b) selecting said polypeptide based on the ability of said polypeptide to activate said luciferase,

wherein said diverse collection and said luciferase are incubated in a diverse collection of biological cells.

In a particular embodiment, said luciferase is Firefly luciferase according to SEQ ID NO: 7.

In contrast to EGFP that has been examined by Xuan et al., as discussed above in the Background section, and which contains a solvent-exposed lysine residue, luciferases, such as Firefly luciferase, contain a lysine residue that is located in the active center of the enzyme. While this residue is essential for the enzymatic activity leading to the bioluminescence, so that blocking of that lysine residue by attachment of protecting groups such as acetyl groups results in the abolishment of the protein's enzymatic activity and thus the bioluminescence, the proper folding of luciferase, particularly Firefly luciferase, does not appear to be hindered by such protecting groups. Surprisingly, the present inventors identified that the blocked essential lysine residue in the active center of the luciferase is still accessible for polypeptides having demodification, in particular deacylation, activity.

In the context of the present invention, the term “luciferase” refers to Firefly luciferase having a protein sequence according to SEQ ID NO: 7, to functional variants thereof and/or to luciferases from other organisms that are oxidoreductases and contain an essential lysine residue in the active center of the enzyme. For the sake of clarity, any reference herein to “residue K529” refers to the lysine in position 529 of the sequences as shown in SEQ ID NO: 7 (see Branchini et al., The role of lysine 529, a conserved residue of the acyl-adenylate-forming enzyme superfamily, in firefly luciferase. Biochemistry 39 (2000) 5433-5440). In the case of variants of Firefly luciferase, or of any luciferase from a different organism (see, for example, Leach, Natural product communications 3 (2008) 1437-1448; Viviani, Cell. Mol. Life Sci. 59 (2002) 1833-1850; Ye et al. Biochimica et Biophysica Acta 1339 (1997) 39-52), the actual position of the essential lysine corresponding to K529 according to SEQ ID NO: 7 may be different. However, the reference to position K529 in the context of the present invention is used synonymously with “the position of the essential lysine in the active center of the enzyme”. Methods for identifying luciferases having an essential lysine in the active center of the enzyme by reviewing the prior art or by analyzing existing luciferases are well known to anyone of ordinary skill in the art.

In a particular embodiment the method of that second aspect further comprises the following counter-screening steps:

(c) incubating a polypeptide selected in step (b) with a luciferase comprising an inactivated residue K529, where said residue is differentially inactivated by a modification different from the modification used in step (a); and

(d) screening said polypeptide based on the inability of said polypeptide to activate said luciferase comprising said differentially inactivated residue K529.

In a third aspect, the present invention relates to a method of screening or selecting a KDAC inhibitor from a diverse collection of putative KDAC inhibitors, wherein the method comprises the following steps:

(a) incubating a polypeptide having a lysine demodification, in particular a lysine deacylation, activity with a member of said diverse collection;

(b) adding a peptide or polypeptide comprising an essential lysine residue inactivated by a modification, in particular an acylation, of said essential lysine residue; and

(c) identifying a KDAC inhibitor by the ability to inhibit the demodification, in particular the deacetylation, activity of said polypeptide,

wherein the KDAC inhibiting activity of said KDAC inhibitor is reciprocal to the activity of said polypeptide to activate the peptide or polypeptide comprising the inactivated essential lysine residue; in particular, wherein the method is performed in a biological cell.

In particular embodiments of the methods according to the first or third aspect, the peptide or polypeptide comprising an essential lysine residue inactivated by a modification is OMP decarboxylase.

In particular embodiments, the OMP decarboxylase is buddying yeast OMP decarboxylase (Ura3) or E. coli pyrF.

In particular embodiments, the OMP decarboxylase is buddying yeast OMP decarboxylase (Ura3) comprising an inactivated residue K93.

In particular embodiments, the peptide or polypeptide an essential lysine residue inactivated by a modification is a luciferase comprising an inactivated residue K529.

In a particular embodiment, said luciferase is Firefly luciferase according to SEQ ID NO: 7.

In particular embodiments, the luciferase comprises an amino acid sequence having at least 90% sequence homology to SEQ ID NO: 7, particularly wherein said luciferase is Firefly luciferase comprising the sequence according to SEQ ID NO: 7.

In particular embodiments of the methods of the present invention, the essential lysine residue is inactivated by acylation or by an alternative protection group, particularly by acylation.

In particular such embodiments, the essential lysine residue is inactivated by acylation with an acyl group selected from the groups of acetyl, crotonyl, tert.-butyloxycarbonyl (Boc), allyloxycarbonyl (Aloc), propargyloxycarbonyl (Poc), benzyloxycarbonyl (Z), 2,2,2-trichloroethyloxycarbonyl (Troc), azidomethoxycarbonyl (Azoc), 2-chlorobenzyloxycarbonyl (Cl—Z) and trifluoroacetyl (tfa).

In particular embodiments of the methods of the present invention, the biological cell is a bacterial cell, in particular wherein the bacterial cell is an E. coli cell.

In particular embodiments, the bacterial cell is an E. coli cell, which lacks a gene encoding pyrF and/or cobB and/or wherein the activity of pyrF and/or cobB is inhibited in said E. coli cell.

In a fourth aspect, the present invention relates to a luciferase, in particular a luciferase comprising an amino acid sequence having at least 90% sequence homology to SEQ ID NO: 7, wherein the polypeptide comprises an inactivated lysine residue at a position corresponding to position 529 of SEQ ID NO: 7.

In a particular embodiment, said polypeptide comprises the sequence according to SEQ ID NO: 7.

In particular embodiments of that fourth aspect, the lysine residue is inactivated by acylation, in particular by acylation with an acyl group selected from the groups of acetyl, crotonyl, tert.-butyloxycarbonyl (Boc), allyloxycarbonyl (Aloc), propargyloxycarbonyl (Poc), benzyloxycarbonyl (Z), 2,2,2-trichloroethyloxycarbonyl (Troc), azidomethoxycarbonyl (Azoc), 2-chlorobenzyloxycarbonyl (Cl—Z) and trifluoroacetyl (tfa).

In particular embodiments the polypeptide additionally comprises a purification tag, particularly a 6×His-tag.

In yet another aspect, the present invention relates to the use of a luciferase according to the present invention in a method for determining and/or measuring the activity of a demodification agent, particularly a deacylation agent, more particularly a deacylation agent, such as a lysine deacetylase, in vivo or in vitro.

In particular embodiments, such method is performed as described in Example 7 below.

In a fourth aspect, the present invention relates to a nucleic acid encoding the polypeptide of the present invention, wherein the codon encoding the essential lysine residue is replaced by an amber stop codon.

In particular embodiments the nucleic acid comprises a nucleic acid sequence having at least 80% sequence homology to SEQ ID NO: 8, wherein the codon encoding the essential lysine residue is replaced by an amber stop codon.

In a particular embodiment, said nucleic acid sequence encodes the protein according to SEQ ID NO: 7.

In a fifth aspect, the present invention relates to a mutant polypeptide comprising an amino acid sequence having at least 98, preferably 99% sequence homology with SEQ ID NOs: 2, 3, 4, 5 or 6 and having lysine demodification, in particular lysine deacylation, activity, wherein the mutant polypeptide is not identical to SEQ ID NO: 1.

As shown in the appended examples, the methods of the present invention surprisingly and unexpectedly result in the identification of KDAC variants that remove typical protection groups for lysine side chains to an extent sufficient to activate an amount of Ura3 enzyme to sustain growth of bacterial cells in the absence of uracil. Such an activity is surprising and unexpected in view of the prior art, which has been unable to provide KDAC variants showing such an improved activity, which allows bacterial cells to grow in the absence of essential growth medium components such as uracil. The mutant polypeptides of the invention, catalyzing bioorthogonal reactions are the key to success for safe prodrug strategies in cancer therapy. Presently, enzymes to activate prodrugs are either of human origin (with the disadvantage of being present in other tissues and therefore causing side effects) or from a different organism (with the disadvantage of being immunogenic). The mutant polypeptides of the invention with bioorthogonal activity evolved from a parent enzyme of human origin combine the advantages of both approaches.

In one embodiment of the invention, the mutant polypeptide comprises a mutation of A37S, Y53W, R56W, 153V and/or V148L with respect to SEQ ID NO: 1. These mutations have been shown to surprisingly and unexpectedly significantly improve the activity of KDAC to an extent as shown herein.

The mutant polypeptide of the invention preferably comprises a sequence identical to any one of SEQ ID NOs: 2, 3, 4, 5 or 6. More preferably, the mutant polypeptide of the invention is identical to any one of SEQ ID NOs: 2, 3, 4, 5 or 6.

Accordingly, the present invention is not restricted to KDAC variants as in any one of SEQ ID NOS: 2, 3, 4, 5 or 6, but extends, in particular, to KDAC variants which are structurally related to any of the above variants such as, e.g., truncated versions thereof. Thus, the present invention also relates to variants of KDAC, which are structurally related to KDAC variants as in any one of SEQ ID NOS: 2, 3, 4, 5 or 6 and which show one or more substitutions and/or deletions and/or insertions. The term “structurally related” refers to KDAC variants, which show a sequence identity of at least n % to the sequence shown in any one of SEQ ID NOS: 2, 3, 4, 5 or 6 with n being between 98 and 100, but not identical to SEQ ID NO: 1.

Thus, in one embodiment the variant according to the present invention has or preferably is derived from a sequence which is at least n % identical to any one of SEQ ID NOS: 2, 3, 4, 5 or 6 with n being between 98 and 100, and it has (a) substitution(s) and/or (a) deletion and/or (an) insertion(s). When the sequences which are compared do not have the same length, the degree of identity either refers to the percentage of amino acid residues in the shorter sequence which are identical to amino acid residues in the longer sequence or to the percentage of amino acid residues in the longer sequence which are identical to amino acid residues in the shorter sequence. Preferably, it refers to the percentage of amino acid residues in the shorter sequence, which are identical to amino acid residues in the longer sequence. The degree of sequence identity can be determined according to methods well known in the art using preferably suitable computer algorithms such as CLUSTAL.

When using the Clustal analysis method to determine whether a particular sequence is, for instance, at least 98% identical to a reference sequence default settings may be used or the settings are preferably as follows: Matrix: BLOSUM 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

Preferably, the degree of identity is calculated over the complete length of the sequence.

Amino acid residues located at a position corresponding to a position as indicated herein-below in the amino acid sequence shown in any one of SEQ ID NOS: 2, 3, 4, 5 or 6 can be identified by the skilled person by methods known in the art. For example, such amino acid residues can be identified by aligning the sequence in question with the sequence shown in SEQ ID NO:1 and by identifying the positions which correspond to the above indicated positions of SEQ ID NO:1. The alignment can be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences.

In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

When the amino acid sequences of the mutant polypeptides are aligned by means of such a method, regardless of insertions or deletions that occur in the amino acid sequences, the positions of the corresponding amino acid residues can be determined in each of the KDAC variants.

In the context of the present invention, “substituted with another amino acid residue” means that the respective amino acid residues at the indicated position can be substituted with any other possible amino acid residues, e.g. naturally occurring amino acids or non-naturally occurring amino acids (Brustad and Arnold, Curr. Opin. Chem. Biol. 15 (2011), 201-210), preferably with an amino acid residues selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Preferred substitutions for certain positions are indicated further below. Moreover, the term “substituted” or “substitution” also means that the respective amino acid residue at the indicated position is modified.

Such modifications include naturally occurring modifications and non-naturally occurring modifications. Naturally occurring modifications include but are not limited to eukaryotic post-translational modification, such as attachment of functional groups (e.g. acetate, phosphate, hydroxyl, lipids (myristoylation of glycine residues) and carbohydrates (e.g. glycosylation of arginine, asparagines etc.). Naturally occurring modifications also encompass the change in the chemical structure by citrullination, carbamylation and disulphide bond formation between cysteine residues; attachment of co-factors (FMN or FAD that can be covalently attached) or the attachment of peptides (e.g. ubiquitination or sumoylation).

Non-naturally occurring modifications include, e.g., in vitro modifications such as biotinylation of lysine residue or the inclusion of non-canonical amino acids (see Liu and Schultz, Annu. Rev. Biochem. 79 (2010), 413-44 and Wang et al., Chem. Bio. 2009 Mar. 27; 16 (3), 323-336; doi:101016/jchembiol.2009.03.001).

In the context of the present invention, “deleted” or “deletion” means that the amino acid at the corresponding position is deleted.

In the context of the present invention, “inserted” or “insertion” means that at the respective position one or two, preferably one amino acid residue is inserted, preferably in front of the indicated position.

In accordance with the foregoing, the present invention relates to a variant of KDAC, wherein the KDAC variant is characterized in that it shows one or more substitutions, deletions and/or insertions in comparison to the corresponding sequence from which it is derived and wherein these substitutions, deletions and/or insertions occur at one or more of the positions corresponding to positions 37, 53, 56, 92 and/or 148 in the amino acid sequence shown in SEQ ID NO:1. Thus, in one embodiment, the invention relates to a mutant polypeptide having a sequence of SEQ ID NO:1 with 1 to 5 amino acid substitutions, preferably at positions 37, 53, 56, 92 and/or 148 and more preferably mutations A37S, Y53W, R56W, I92V and/or V148L.

In even more preferred embodiments, the variant according to the invention showing an improved activity in demodification, in particular lysine deacylation, of an essential lysine residue is characterized in that it has multiple mutations. As it is exemplified in the examples further below, variants have been found bearing multiple mutations which exhibit an increase in the reaction rate of the conversion of a modified essential lysine residue to the unmodified lysine. These variants bearing multiple mutations are summarized in the following. Accordingly, in a very preferred embodiment, the variant according to the invention is characterized in that it comprises deletions, substitutions and/or insertions wherein the deletions/insertions/substitutions are at positions 37, 53, 56, 92 and 148 in the amino acid sequence shown in SEQ ID NO:1 or at positions corresponding to these positions. Preferably, such a variant has the following substitutions in the amino acid sequence shown in SEQ ID NO:1 or at positions corresponding to these positions: A37S, Y53W, R56W, I92V and V148L.

Conservative substitutions of peptides/polypeptides, which may furthermore be part of the mutant polypeptides of the invention, are shown below.

Ala (A) Val; Leu;

Arg (R) Lys; His

Asn (N) Gln; His; Asp, Lys; Arg

Asp (D) Glu; Asn

Cys (C) Ser; Ala

Gln (Q) Asn; Glu

Glu (E) Asp; Gln

Gly (G) Ala

His (H) Asn; Gln; Lys; Arg

He (I) Leu; Val; Met; Ala; Phe; Norleucine

Leu (L) Norleucine; Ile; Val; Met; Ala; Phe

Lys (K) Arg; Gln; Asn

Met (M) Leu; Phe; Ile

Phe (F) Trp; Leu; Val; Ile; Ala; Tyr

Pro (P) Ala

Ser (S) Thr

Thr (T) Val; Ser

Trp (W) Tyr; Phe

Tyr (Y) Trp; Phe; Thr; Ser

Val (V) Ile; Leu; Met; Phe; Ala; Norleucine

Amino acids may be grouped according to common side-chain properties:

(1) hydrophobic: Norleucine, Met, Ala, Val, Leu, Ile

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Amino acids may also be grouped according to common side-chain size, for example, small amino acids (Gly, Ala, Ser, Pro, Thr, Asp, Asn), or bulky hydrophobic amino acids (Met, Ile, Leu). Substantial modifications in the biological properties of the peptide/polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will entail exchanging a member of one of these classes for another class.

The KDAC variants of the invention have an improved activity of demodification, in particular lysine deacylation, of lysine as compared to the unmodified KDAC polypeptide as shown in SEQ ID NO: 1. In this respect, an “improved activity of demodification, in particular lysine deacylation, of lysine” or similar terms as used herein, can be determined by, for example, methods using a luciferase, particularly Firefly luciferase, with modifications on lysine-529. Specifically, demodification, in particular lysine deacylation, activity can be determined using an assay where the KDAC variant of the invention is incubated with the modified luciferase directly in a whole cell lysate and activity is compared to the activity of wild-type KDAC, in particular cobB. Additionally or alternatively, activities of KDAC variants can be assayed using the bacterial system described further below. Both tests have been surprisingly and unexpectedly shown to provide comparable results (FIG. 3).

In a further embodiment, the present invention relates to a nucleic acid molecule encoding the KDAC variant of the invention. Moreover, the present invention relates in a further embodiment to a vector comprising said nucleic acid. Further, in yet another embodiment, the present invention relates to a host cell comprising said vector. The embodiments relating to the nucleic acid, the vector and the host cell of the present invention are further described in the following in more detail.

A KDAC variant of the present invention can be fused to a homologous or heterologous polypeptide or protein, an enzyme, a substrate or a tag to form a fusion protein. Fusion proteins in accordance with the present invention will have the same improved activity as the KDAC variant of the present invention. Polypeptides, enzymes, substrates or tags that can be added to another protein are known in the art. They may be useful for purifying or detecting the proteins of the invention. For instance, tags that can be used for detection and/or purification are e.g. FLAG-tag, His6-tag or a Strep-tag. Alternatively, the protein of the invention can be fused to an enzyme e.g. luciferase, for the detection or localisation of said protein. Other fusion partners include, but are not limited to, bacterial β-galactosidase, trpE, Protein A, β-lactamase, alpha amylase, alcohol dehydrogenase or yeast alpha mating factor. It is also conceivable that the polypeptide, enzyme, substrate or tag is removed from the protein of the invention after e.g. purification. Fusion proteins can typically be made by either recombinant nucleic acid methods or by synthetic polypeptide methods known in art.

The present invention further relates to a nucleic acid molecule encoding a KDAC variant of the present invention and to a vector comprising said nucleic acid molecules. Vectors that can be used in accordance with the present invention are known in the art. The vectors can further comprise expression control sequences operably linked to the nucleic acid molecules of the present invention contained in the vectors. These expression control sequences may be suited to ensure transcription and synthesis of a translatable RNA in bacteria or fungi. Expression control sequences can for instance be promoters. Promoters for use in connection with the nucleic acid molecules of the present invention may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

Polynucleotide,” or “nucleic acid,” as used interchangeably herein, refer to polymers of nucleotides of any length, and include, but are not limited to, DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR, CO or CH.sub.2 (“formacetal”), in which each R or R is independently H or substituted or unsubstituted alkyl (1-20 C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The polynucleotide(s) of the present invention may be part of a vector. Preferably, the vector of the present invention is an expression vector. Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, N.Y., (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-ß-D-thiogalactopyranoside) (deBoer et al., Proc. Natl. Acad. Sci. USA 80 (1983), 21-25). Termination signals for transcription are also described in the literature.

In addition, the present invention relates to a host cell comprising the vector of the present invention.

In a preferred embodiment, the host cell according to the presenting invention is a microorganism, in particular a bacterium or a fungus. In a more preferred embodiment, the host cell of the present invention is E. coli, a bacterium of the genus Clostridium or a yeast cell, such as S. cerevisiae. In another preferred embodiment the host cell is a plant cell or a non-human animal cell.

The transformation of the host cell with a vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc.

In one preferred embodiment, the organism according to the present invention which can be employed in the method according to the invention is an organism, preferably a microorganism, which lacks the capacity to produce an essentially required factor for growth. For example, the organism, preferably the microorganism, may lack the capacity to produce essential amino acid(s) or nucleobase(s). This is preferably achieved by deleting or otherwise modifying one or more enzymes necessary for the production of the said factor, e.g. enzymes converting precursors of such actors to the ultimately essential factor. One example within the meaning of the present invention is Ura3, which is necessary to produce uracil. The enzyme that is modified/inactivated carries an essential lysine residue, which is modified/inactivated by modifying the essential lysine residue. Expression of the mutant polypeptide of the invention may then convert the inactivated enzyme to its active form. Conversion then allows the organism, preferably the microorganism, to produce the said essential factor so that all components necessary for growth are present. In a preferred embodiment of the invention, the host cell, preferably the microorganism, lacks a gene encoding pyrF and/or cobB. Such a selection system can be used to identify a KDAC variant, i.e. a mutant polypeptide of the invention, with the ability to revert the modification of the lysine residue in a pool of inactive mutants.

In such an embodiment, the organism according to the invention is an organism, preferably a microorganism, which lacks a gene encoding pyrF and/or cobB and which is recombinant in the sense that it has further been genetically modified so as to express a mutant polypeptide according to the present invention. Thus, the term “recombinant” means that the organism is genetically modified so as to contain a foreign nucleic acid molecule encoding a KDAC variant enzyme of the present invention as defined above. The term “foreign” in this context means that the nucleic acid molecule does not naturally occur in said organism/microorganism. This means that it does not occur in the same structure or at the same location in the organism/microorganism. In one preferred embodiment, the foreign nucleic acid molecule is a recombinant molecule comprising a promoter and a coding sequence encoding the KDAC variant, in which the promoter driving expression of the coding sequence is heterologous with respect to the coding sequence. Heterologous in this context means that the promoter is not the promoter naturally driving the expression of said coding sequence but is a promoter naturally driving expression of a different coding sequence, i.e., it is derived from another gene, or is a synthetic promoter or a chimeric promoter. Preferably, the promoter is a promoter heterologous to the organism/microorganism, i.e. a promoter which does not naturally occur in the respective organism/microorganism. Even more preferably, the promoter is an inducible promoter. Promoters for driving expression in different types of organisms, in particular in microorganisms, are well known to the person skilled in the art.

In another preferred embodiment the nucleic acid molecule is foreign to the organism/microorganism in that the encoded KDAC variant, is/are not endogenous to the organism/microorganism, i.e. are naturally not expressed by the organism/microorganism when it is not genetically modified.

The term “recombinant” in another embodiment means that the organism is genetically modified in the regulatory region controlling the expression of an enzyme as defined above which naturally occurs in the organism so as to lead to an increase in expression of the respective enzyme in comparison to a corresponding non-genetically modified organism. Such a modification of a regulatory region can be achieved by methods known to the person skilled in the art. One example is to exchange the naturally occurring promoter by a promoter which allows for a higher expression or to modify the naturally occurring promoter so as to show a higher expression. Thus, in this embodiment the organism contains in the regulatory region of the gene encoding an enzyme as defined above a foreign nucleic acid molecule which naturally does not occur in the organism and which leads to a higher expression of the enzyme in comparison to a corresponding non-genetically modified organism.

The foreign nucleic acid molecule may be present in the organism/microorganism in extrachromosomal form, e.g. as plasmid, or stably integrated in the chromosome. A stable integration is preferred.

Methods for preparing the above mentioned genetically modified organism, preferably microorganisms, are well known in the art. Thus, generally, the organism/microorganism is transformed with a DNA construct allowing expression of the respective enzyme in the microorganism. Such a construct normally comprises the coding sequence in question linked to regulatory sequences allowing transcription and translation in the respective host cell, e.g. a promoter and/enhancer and/or transcription terminator and/or ribosome binding sites etc.

The mutant polypeptide of the invention may be used in therapy. In this respect, the mutant polypeptides of the invention may preferably be combined, either in one or separate formulations, with a peptide or polypeptide comprising an inactive essential lysine residue for use in treating cancer. The invention also provides for therapy of diabetes and/or neurodegenerative diseases using the means provided herein. The mutant polypeptide of the invention may also be used against symptoms related to aging by, e.g., being used in methods for screening of KDAC activity modulating compounds.

The term “peptide” generally refers to a contiguous and relatively short sequence of amino acids linked by peptidyl bonds. Typically, but not necessarily, a peptide has a length of about 2 to 50 amino acids, 4-40 amino acids or 10-30 amino acids. Although the term “polypeptide” generally refers to longer forms of a peptide, the two terms can be and are used interchangeably in some contexts herein.

The terms “amino acid” and “residue” are used interchangeably herein. A “region” of a polypeptide is a contiguous sequence of 2 or more amino acids. In other embodiments, a region is at least about any of 3, 5, 10, 15 contiguous amino acids.

In one embodiment, the inactivated lysine residue of the peptide or polypeptide of the invention comprising an essential lysine residue is acylated, in particular acetylated, or comprises an alternative protection group.

Within the present invention, the term “acetylation” describes a reaction that introduces an acetyl functional group into a chemical compound. “Deacetylation” is the removal of an acetyl group.

Acetylation refers to the process of introducing an acetyl group (resulting in an acetoxy group) into a compound, namely the substitution of an acetyl group for an active hydrogen atom. A reaction involving the replacement of the hydrogen atom of a hydroxyl group with an acetyl group (CH₃CO) yields a specific ester, the acetate. Acetic anhydride is commonly used as an acetylating agent reacting with free hydroxyl groups. For example, it is used in the synthesis of aspirin, heroin, and THC-O-acetate.

Proteins are typically acetylated on lysine residues and this reaction relies, in vivo, on acetyl-coenzyme A. However, proteins can also artificially be acetylated. In histone acetylation and deacetylation, histone proteins are acetylated and deacetylated on lysine residues in the N-terminal tail as part of gene regulation. The regulation of transcription factors, effector proteins, molecular chaperones, and cytoskeletal proteins by acetylation and deacetylation is a significant post-translational regulatory mechanism. These regulatory mechanisms are analogous to phosphorylation and dephosphorylation by the action of kinases and phosphatases. Not only can the acetylation state of a protein modify its activity but there has been recent suggestion that this post-translational modification may also crosstalk with phosphorylation, methylation, ubiquitination, sumoylation, and others for dynamic control of cellular signaling.

If an essential lysine residue, i.e. a lysine residue required for the natural activity of the acetylated polypeptide, is acetylated, or more generally acylated or otherwise modified by covalent binding of a moiety to the lysine residue, it will in some cases loose its activity or show a reduced activity. Therefore, the peptide or polypeptide comprising an essential lysine residue of the invention is named “inactive” due to the acylation or modification. In this respect, “inactive” means that the peptide or polypeptide does not show its natural activity to the same extent as in its “active” form, i.e. without being acylated or otherwise modified at the essential lysine residue. The activity may be reduced due to acylation or modification from 100% to 90%, 80%, 70%, 60%, 50%, 40%, 30%, 20%, 10% or even 0%. It is preferred that the activity is reduced to a minimum.

The essential lysine residue of the peptide or polypeptide comprising an essential lysine residue of the invention may also be inactivated by alternative protection groups. Such protection groups are generally known in the art and every protection group is possible as long as it can be removed by the mutant polypeptide having lysine demodification, in particular lysine deacylation, activity of the invention. In the case of deacylation, such protection groups may be N(ε)-tert.-butyloxycarbonyl (Boc), N(ε)-allyloxycarbonyl (Aloc), N(ε)-propargyloxycarbonyl (Poc), N(ε)-benzyloxycarbonyl (Z), N(ε)-2,2,2-trichloroethyloxycarbonyl (Troc), N(ε)-azidomethoxycarbonyl (Azoc), N(ε)-2-chlorobenzyloxycarbonyl (Cl—Z) or N(ε)-trifluoroacetyl (tfa).

In the context of the present invention, the term “acyl” is used as defined by IUPAC as a group formed by removing one or more hydroxy groups from oxoacids that have the general structure RₖE(═O)ₗ(OH)ₘ (with l being different from 0), and replacement analogues of such acyl groups. Thus, the term “acyl” as used herein includes an oxycarbonyl group R—O—(C═O)—, which can be regarded as being derived from the oxoacid carbonic acid C(═O)(OH)₂ with E being C; k being 0; l being 1; and m being 2.

The invention furthermore relates to a method of screening for a mutant polypeptide having lysine demodification, in particular lysine deacylation, activity, wherein the method comprises the following steps (a) incubating a mutant polypeptide having an amino acid sequence with at least 80% sequence identity to SEQ ID NO: 1 with a peptide or polypeptide comprising an inactivated essential lysine residue; and (b) determining the activity of the mutant polypeptide to activate the peptide or polypeptide comprising the inactivated essential lysine residue, wherein the mutant polypeptide and the peptide or polypeptide comprising an inactivated essential lysine residue are incubated in a biological cell.

Accordingly, a selection system for KDACs with altered substrate specificity and/or reactivity against bioorthogonal chemical protection groups is reported. The system builds on the incorporation of lysine derivatives by genetic code expansion in reporter enzymes with essential active site lysine residues. The reporter enzyme containing the lysine derivative is an inactive precursor that is turned on upon removal of the modification, thereby coupling deacetylase activity to a selectable output. This enables to evolve KDACs selective for particular lysine acylations and other bioorthogonal modifications. These KDAC variants may be used to partially complement KDAC deletion strains or to design a prodrug strategy for cancer therapy.

The invention is based on a selection system for lysine deacetylases (KDACs) based on a selectable marker that contains an essential lysine residue. By replacing this residue with modified forms of lysine (e.g. acylated forms, for example acetylated forms, or forms modified by protection with alternative protection groups) using genetic code expansion, we generate an inactive precursor enzyme. Cells must revert the modification to activate the selectable marker, hence coupling KDAC activity to cell survival. Using this system, KDAC variants with increased substrate specificity or the ability to remove protection groups from lysine residues could be created and are provided herein.

Here, the directed evolution of KDACs towards particular acyl substrates and bioorthogonal lysine modifications using a bacterial selection system is reported. The new polypeptides of the invention can be used for partial complementation of KDAC deletion strains to reveal the physiological role of particular lysine acylations. Bioorthogonal “eraser” enzymes facilitate the activation of pro-peptides or pro-enzymes by removing protection groups installed on lysine residues. These bioorthogonal “eraser” enzymes may therefore find applications in prodrug strategies of cancer therapy.

The herein described KDAC assay can also be used to screen for KDAC inhibitors. Such methods comprise an additional step of adding a small chemical molecule and determining whether said chemical molecule is able to inhibit the activity of the KDAC polypeptide to activate the polypeptide comprising the essential lysine residue. In one embodiment, the invention thus relates to a method of screening for KDAC inhibitors, wherein the method comprises (a) incubating a polypeptide having an amino acid sequence with at least 80% sequence identity to SEQ ID NO: 1 and having deacetylation activity with a small molecule; (b) adding a peptide or polypeptide comprising an inactivated essential lysine residue; and (c) determining the activity of the mutant polypeptide to activate the peptide or polypeptide comprising the inactivated essential lysine residue, wherein the KDAC inhibiting activity of the small molecule is reciprocal to the activity of the mutant polypeptide to activate the peptide or polypeptide comprising the inactivated essential lysine residue. In a preferred embodiment, a library of small chemical molecules is screened by repeating the method for each member of said library.

The screening method of the invention may be carried out in any biological cell, preferably a bacterial cell. Accordingly, in one embodiment, the invention relates to a method of screening for a mutant polypeptide having lysine demodification, in particular lysine deacylation, activity, wherein the method comprises the following steps (a) incubating a mutant polypeptide having an amino acid sequence with at least 80% sequence identity to SEQ ID NO: 1 with a peptide or polypeptide comprising an inactivated essential lysine residue; and (b) determining the activity of the mutant polypeptide to activate the peptide or polypeptide comprising the inactivated essential lysine residue, wherein the mutant polypeptide and the peptide or polypeptide comprising an inactivated essential lysine residue are incubated in a bacterial cell. However, the screening method of the invention is not limited to sequences having 80% identity to SEQ ID NO:1. That is, the starting sequence does not have to be related to CobB, which is an example of sirtuins. The method of the invention can also be based on alternative sequences, for example, starting from HDAC8 or other zinc dependent enzymes.

The bacterial cell is preferably E. coli. In order to determine the activity of the mutant polypeptide having lysine demodification, in particular lysine deacylation, activity, it is preferred that the E. coli cell lacks a gene encoding for pyrF and/or cobB. This is because lysine demodification, in particular lysine deacylation, activity of the mutant polypeptide to be screened can then surprisingly and unexpectedly well correlated with the activity of the mutant polypeptide to be screened. In a preferred embodiment, the mutant polypeptide is not identical to SEQ ID NO:1.

In order to provide a screening method, which can surprisingly and unexpectedly well determine the lysine demodification, in particular lysine deacylation, activity of a mutant polypeptide to be screened, a reporter gene is used, which leads to a detectable and quantifiable signal. In this respect, the skilled person can select reporter genes as long as said reporter gene carries an essential lysine residue, which can be modified and subsequently demodified by the mutant polypeptide of interest. It is preferred that the peptide or polypeptide comprising an inactivated essential lysine residue is OMP decarboxylase or a luciferase, particularly Firefly luciferase. In this respect, it is preferred that OMP decarboxylase is buddying yeast OMP decarboxylase (Ura3) or E. coli pyrF. The essential lysine residue carried by the reporter gene can be inactivated by acetylation or an alternative protection group, as described further above.

In a particular preferred embodiment, the polypeptide comprising an inactivated essential lysine residue is a luciferase, particularly Firefly luciferase, comprising an acylated lysine residue at a position corresponding to K529. In this respect, it has been surprisingly and unexpectedly found that The luciferase-based KDAC assay of the invention has very low production costs. Specifically, typical commercial KDAC assays such as the SIRT-Glo assay (Promega) or the Fluorimetric HDAC Assay Kit (Sigma) are sold at a price amounting to about 2000 times the production costs of the assay of the invention. Moreover, it has been surprisingly found that the methods provided herein using the modified luciferase, particularly Firefly luciferase, have improved sensitivity and a broader dynamic range. In this respect, the method of the invention was compared to the widely used Fluor-de-Lys assay to measure activity of SirT2 (FIG. 4) and CobB (FIG. 5). For both KDACs the methods of the invention using the modified luciferase was able to detect KDAC activity at least at one order of magnitude lower concentrations. The dynamic range of the methods of the invention using the modified luciferase covers 2-3 orders of magnitude. The methods of the invention using the modified luciferase are insensitive to auto-fluorescence. Fluor-de-Lys assays are fluorimetric and therefore unsuited to assay auto-fluorescent molecules for their impact on KDAC activity. Since the methods of the invention using the modified luciferase detect luminescence, auto-fluorescence is unproblematic. In general, the read-out of the methods of the invention using the modified luciferase is orthogonal to the Fluor-de-Lys assay and therefore ideally suited as a second screening method for KDAC effectors. The methods of the invention using the modified luciferase are more convenient and faster. Fluor-de-Lys and similar assays are based on a two-step reaction that involves proteolytic cleavage of the deacylated substrate in the second reaction step. This hinders the development of continuous assays because the protease also inactivates any KDAC. The methods of the invention using the modified luciferase can be used in a continuous format. Fluor-de-Lys assays are typically performed for 2 h at 37° C. during the first step. The methods of the invention using the modified luciferase are much faster, typical reaction times are 15-30 min at 25° C. Luminescence can be measured immediately (or simultaneously), while Fluor-de-Lys assays require 20 min at 37° C. incubation during the second step (proteolytic digest). Hence, the methods of the invention using the modified luciferase are up to ten times faster than the Fluor-de-Lys assay. The methods of the invention using the modified luciferase can be used to measure the effect of inhibitors on KDAC activity. Nicotinamide was titrated into the deacetylation reaction of SirT2 (FIG. 6). The dose-dependent inhibition that was observed indicates an IC₅₀ for the reaction of approximately 30 μM, which is within the range of published values, thus, showing the reliability of the methods of the invention. Thus, in one embodiment, the invention relates to a method of screening for a mutant polypeptide having lysine demodification, in particular lysine deacylation, activity, wherein the method comprises the following steps (a) incubating a mutant polypeptide having an amino acid sequence with at least 80% sequence identity to SEQ ID NO: 1 with a polypeptide comprising an inactivated essential lysine residue; and (b) determining the activity of the mutant polypeptide to activate the polypeptide comprising the inactivated essential lysine residue, wherein the mutant polypeptide and the polypeptide comprising an inactivated essential lysine residue are incubated in a biological cell and wherein the polypeptide comprising an inactivated essential lysine residue is a luciferase, particularly Firefly luciferase, comprising an acylated lysine, preferably at residue 529.

Thus, in one embodiment, the invention relates to the methods of the invention, wherein the essential lysine residue that leads to inactivation of the polypeptide is acylated, particularly acetylated, and is residue K529 of luciferase, particularly Firefly luciferase. In a preferred embodiment, the luciferase comprises an amino acid sequence having at least 90% sequence homology to SEQ ID NO: 7. In this context, SEQ ID NO: 7 relates to the commonly used Firefly luciferase carrying an acylated, particularly acetylated lysine residue at position 529. The skilled person understands that variants of this sequence will show identical or similar activity and thus may also be used in the present invention provided that the lysine residue corresponding to the residue 529 of SEQ ID NO: 7 is acylated.

In a further embodiment, the invention relates to a polypeptide comprising an amino acid sequence having at least 90% sequence homology to SEQ ID NO: 7, wherein the polypeptide comprises a modified lysine residue at a position corresponding to position 529 of SEQ ID NO: 7. Said modification may be an acetylation, crotonylation, butyrylation, propionylation, 2-hydroxybutyrylation or acylation by a group such as Boc or Aloc. Preferably, the modification is acetylation. In a preferred embodiment, the polypeptide additionally comprises a purification tag, preferably a 6×His-tag.

The invention also relates to a nucleic acid encoding the polypeptide of the invention. It is preferred that the nucleic acid of the invention comprises a nucleic acid sequence having at least 80% sequence homology to SEQ ID NO: 8.

The polypeptide and/or nucleic acid of the invention may be provided in form of a kit, wherein the kit preferably also comprises instructions with respect to the methods of the invention. The polypeptide and nucleic acid are thus also provided for use in a method of the invention.

The invention furthermore relates to devices for carrying out the screening method, in particular devices used for high-throughput screening.

The invention also relates to an E. coli strain lacking expression of pyrF and cobB. Preferably, the the E. coli strain of the invention expresses Ura3 comprising a modified essential lysine residue.

The invention also relates to a kit comprising the E. coli strain of the invention and/or the mutant polypeptide of the invention.

The present invention also relates to the following items:
1. A mutant polypeptide comprising an amino acid sequence having at least 99% sequence homology with SEQ ID NOs: 2, 3, 4, 5 or 6 and having lysine demodification activity, wherein the mutant polypeptide is not identical to SEQ ID NO: 1.
2. The mutant polypeptide of item 1, wherein the mutant polypeptide comprises an amino acid sequence having 1 to 5 mutations in the amino acid sequence of SEQ ID NO: 1.
3. The mutant polypeptide of item 1 or 2, wherein the mutant polypeptide comprises one or more mutation(s) at positions 37, 53, 56, 92 and/or 148 of SEQ ID NO:1.
4. The mutant polypeptide of item 3, wherein the polypeptide comprises mutations A37S, Y53W, R56W, I92V and/or V148L with respect to SEQ ID NO: 1.
5. The mutant polypeptide of any one of items 1 to 4, which is SEQ ID NO: 2, 3, 4, 5 or 6.
6. The mutant polypeptide of item 1 and a peptide or polypeptide comprising an inactivated essential lysine residue for use in treating cancer.
7. The mutant polypeptide and the peptide or polypeptide for use of item 6, wherein the mutant polypeptide and the peptide or polypeptide are administered together or sequentially.
8. The mutant polypeptide and the peptide or polypeptide for use of item 6 or 7, wherein the inactivated lysine residue of the peptide or polypeptide is acylated, in particular acetylated, or comprises an alternative protection group.
9. A method of screening for a mutant polypeptide having lysine demodification activity, wherein the method comprises the following steps:
   (a) incubating a mutant polypeptide having an amino acid sequence with at least 80% sequence identity to SEQ ID NO: 1 with a peptide or polypeptide comprising an inactivated essential lysine residue; and
   (b) determining the activity of the mutant polypeptide to activate the peptide or polypeptide comprising the inactivated essential lysine residue,
   wherein the mutant polypeptide and the peptide or polypeptide comprising an inactivated essential lysine residue are incubated in a biological cell.
10. A method of screening for KDAC inhibitors, wherein the method comprises the following steps:
   (a) incubating a polypeptide having an amino acid sequence with at least 80% sequence identity to SEQ ID NO: 1 and having deacetylation activity with a small molecule;
   (b) adding a peptide or polypeptide comprising an inactivated essential lysine residue; and
   (c) determining the activity of the mutant polypeptide to activate the peptide or polypeptide comprising the inactivated essential lysine residue,
   wherein the KDAC inhibiting activity of the small molecule is reciprocal to the activity of the mutant polypeptide to activate the peptide or polypeptide comprising the inactivated essential lysine residue.
11. The method of item 10, wherein the method is performed in a biological cell.
12. The method of item 9 or 11, wherein the biological cell is a bacterial cell.
13. The method of item 12, wherein the bacterial cell is E. coli.
14. The method of item 12 or 13, wherein the bacterial cells, preferably E. coli cells, lack a gene encoding pyrF and/or cobB.
15. The method of any one of items 9 to 14, wherein the mutant polypeptide has an amino acid sequence that is not identical to SEQ ID NO:1.
16. The method of any one of items 9 to 15, wherein the peptide or polypeptide comprising an inactivated essential lysine residue is OMP decarboxylase or Firefly luciferase.
17. The method of item 16, wherein OMP decarboxylase is buddying yeast OMP decarboxylase (Ura3) or E. coli pyrF.
18. The method of any one of items 9 to 17, wherein the essential lysine residue is inactivated by acylation, in particular acetylation, or by an alternative protection group.
19. The method of items 18, wherein the acetylated essential lysine residue is residue K529 of Firefly luciferase.
20. The method of items 19, wherein the Firefly luciferase comprises an amino acid sequence having at least 90% sequence homology to SEQ ID NO: 7.
21. A polypeptide comprising an amino acid sequence having at least 90% sequence homology to SEQ ID NO: 7, wherein the polypeptide comprises an inactivated lysine residue at a position corresponding to position 529 of SEQ ID NO: 7.
22. The polypeptide of items 21, wherein the lysine residue is inactivated by acetylation.
23. The polypeptide of item 21, additionally comprising a purification tag, preferably a 6×His-tag.
24. A nucleic acid encoding the polypeptide of item 21.
25. The nucleic acid of item 24 comprising a nucleic acid sequence having at least 80% sequence homology to SEQ ID NO: 8.
26. An E. coli strain lacking expression of pyrF and cobB.
27. The E. coli strain of item 26 expressing Ura3 comprising a modified essential lysine residue.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1563), and Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention. A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### FIGURES

FIG. 1: E. coli producing Ura3 K93ac as the sole source of OMP decarboxylase depend on KDAC activity. E. coli DB6566 (ΔpyrF) expressing plasmids to encode wild-type Ura3, Ura3 K93ac or K93boc were plated on agar plates with or without uracil, 5-FOA and the corresponding unnatural amino acid. Nicotinamide (NAM) was added to inhibit endogenous CobB.

FIG. 2: CobB library design. Highlighted amino acid residues were randomized to all possible combinations of natural amino acids to generate a library of thirty million mutants in the active site of CobB.

FIG. 3: Evolved CobB variants can activate Firefly luciferase modified at K529. Dual luciferase reporter (DLR) assays were performed with E. coli producing DLR with the indicated modification of K529 in the Firefly enzyme. Activities were normalized to genetically fused Renilla luciferase and plotted relative to the activity observed for wild-type CobB on the same substrate. A) CobB mutant able to discriminate crotonyl-over acetyl-lysine. B) CobB mutants active against protected forms of lysine.

FIG. 4: Quantification of SirT2 activity using Fluor-de-Lys and FLuc assays. Deacetylation assays were performed at various concentrations of SirT2 using either 10 μM Fluor-de-Lys peptide or 30 nM acetylated FLuc under otherwise identical conditions. KDAC activities were measured by fluorescence (ex. 355/em. 460 nm) or luminescence and normalized to the activity at the highest SirT2 concentration after background subtraction. Luminescence was measured in endpoint or continuous format. The shaded areas indicate respective linear response ranges. Error bars are standard deviation of the means from triplicate measurements.

FIG. 5: Quantification of CobB activity using Fluor-de-Lys and FLuc assays. Deacetylation assays were performed at various concentrations of CobB using either 10 μM Fluor-de-Lys peptide or 30 nM acetylated FLuc under otherwise identical conditions. KDAC activities were measured by fluorescence (ex. 355/em. 460 nm) or luminescence and normalized to the activity at the highest CobB concentration. Luminescence was measured in endpoint format. Error bars are standard deviation of the means from triplicate measurements.

FIG. 6: Inhibition of SirT2 by nicotinamide. Acetylated FLuc (30 nM) was deacetylated with SirT2 (8 μg/ml) in the presence of 1 mM NAD⁺ and increasing concentrations of nicotinamide. Error bars are standard deviations of the means from three independent reactions. IC₅₀ for nicotinamide inhibition of SirT2 is approximately 30 μM.

FIG. 7: FLuc K529ac activation by different KDACs. Sirtuins were used at 20 μg/ml, while HDAC8 concentration was 10 mg/ml with tenfold higher FLuc K529ac. All error bars are standard deviations of triplicates.

FIG. 8: Effectors identified in FLuc K529ac-based screen. A) Inhibitors of SirT2 (IC₅₀ of 1-7 [μM]: 3.2; 5.5; 17; >30; 13; 15; 11. n=2) B) SirT2 activator (1.6 fold at 30 μM) C) Firefly luciferase inhibitor (IC₅₀: 3 μM).

### EXAMPLES

### Example 1—Design of a Selection System for Lysine Deacetylases

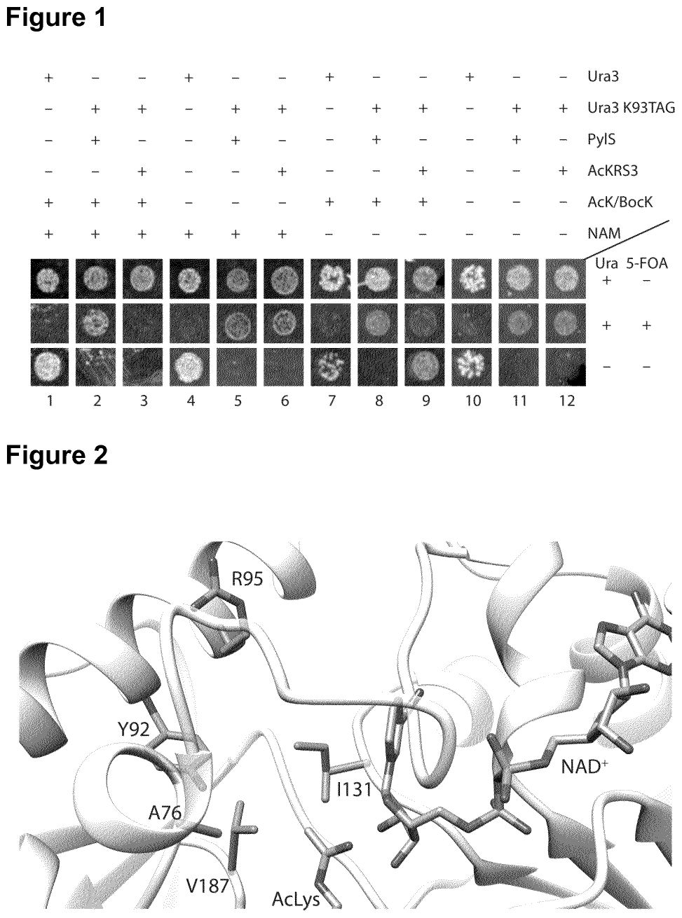

To develop a selection system for KDACs, enzymes with lysine residues essential for activity were searched. Two of these enzymes were tested, orotidine-5′-phosphate (OMP) decarboxylase and firefly luciferase. Both proved to be suitable as selectable marker and reporter enzyme, respectively. When N(ε)-acetyl-lysine was incorporated in place of K93 of budding yeast OMP decarboxylase (Ura3), the protein was unable to support growth of E. coli cells lacking pyrF (the homologue of Ura3) and cobB (the major lysine deacetylase of E. coli, inhibited with nicotinamide) in the absence of uracil (FIG. 1, column 3, bottom). In the presence of cobB, robust growth was observed on minimal medium without uracil, indicating that cobB was able to remove the acetyl group from the active site lysine of Ura3 K93ac. Growth of the same cells is inhibited when 5-fluoro-orotic acid, a compound converted into a toxic metabolite by Ura3, is added to the medium. Hence, this system is able to positively and negatively select E. coli harboring an active lysine deacetylase. The same system can also be used to select for HDAC8 activity, a mammalian class I lysine deacetylase structurally and mechanistically distinct from the sirtuin family member cobB.

Firefly luciferase contains an essential lysine residue (K529) in the active site. Replacing this residue by genetic code expansion with N(ε)-acetyl-lysine rendered the enzyme inactive in the absence of lysine deacetylase cobB. In the presence of cobB, robust activity of the enzyme was observed. Hence, K529ac firefly luciferase can be used to screen for lysine deacetylase activity in E. coli.

### Example 2—Creation of cobB Mutant Libraries

Next, a mutant library was created by randomizing five active site residues (A37, Y53, R56, I92 and V148) of E. coli cobB to all possible combinations of natural amino acids, thereby creating 20⁵ (3.2×10⁶) different mutants (FIG. 2).

### Example 3—Isolation of Acyl-Type Specific Deacetylases

To identify cobB mutants selectively removing crotonyl but not acetyl groups, the library was subjected to two rounds of selection, positive and negative. Therefore, E. coli DH10B ΔpyrF ΔcobB harbouring a reporter plasmid encoding ura3 K93TAG together with wildtype MbPyIRS and the cognate amber suppressor tRNA MbPyIT was transformed with the cobB mutant library. The cells were challenged to grow in the presence of N(ε)-crotonyl-lysine on medium without uracil to select clones able to decrotonylate Ura3 K93cr. Library plasmids were isolated from the pool of surviving clones and used to transform DH10B ΔpyrF ΔcobB harbouring a reporter plasmid encoding AcKRS3 (M. barkeri PyIRS variant specific for N(ε)-acetyl-lysine) instead of MbPyIRS. Cells were grown on plates containing N(ε)-acetyl-lysine and 5-fluoro-orotic acid (5-FOA), which is toxic to cells in the presence of active Ura3, to select against clones able to remove acetyl groups from Ura3 K93ac. The library member-encoding plasmids of the clones surviving the negative selection were isolated and re-transformed into E. coli DH10B ΔpyrF ΔcobB harbouring a reporter plasmid encoding ura3 K93TAG together with wildtype MbPyIRS and the cognate amber suppressor tRNA MbPyIT, and individual clones were arrayed and tested for the ability to survive on medium without uracil in the presence of N(ε)-crotonyl-lysine. Thereby several mutants of CobB were identified that were able to selectively cleave crotonyl, but not acetyl groups off lysine side chains.

### Example 4—Selection of Bioorthogonal Eraser Enzymes

Next, the same cobB mutant library was challenged to remove chemical protection groups from lysine residues. N(ε)-tert.-butyl-oxycarbonyl-lysine (BocK), N(ε)-allyl-oxycarbonyl-lysine (AlocK) and N(ε)-propargyl-oxycarbonyl-lysine (PrK) can be incorporated in proteins using wild-type PyIRS/PyIT. E. coli DH10B ΔpyrF ΔcobB harbouring the mutant library was challenged to grow in the absence of uracil while incorporating one of these unnatural amino acids in Ura3 in place of K93. Surviving clones were arrayed and plasmids isolated from cells that grew in the absence of uracil depending on the presence of one of the unnatural amino acids. Several mutants capable of cleaving AlocK were identified and a single mutant with activity against BocK (Table 1). Individual testing of mutants isolated in the BocK and AlocK selections for activity against PrK revealed several mutants with basal activity.

### Example 5—Quantitative Analysis of Mutant Activities Using Firefly Luciferase Assay

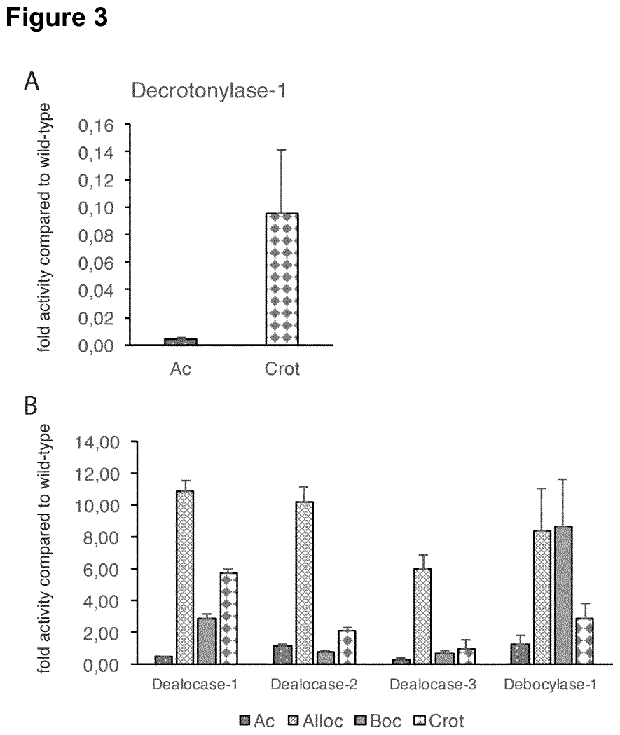

The mutants isolated in the selections were tested using Firefly dual luciferase assays. E. coli DH10B ΔpyrF ΔcobB were transformed with plasmids expressing Firefly luciferase with the relevant modification on lysine-529 and the cobB mutants. Luciferase activity was tested directly in whole cell lysates and compared to the activity of wild-type cobB towards the modifications. The activities observed for the evolved KDAC variants correlated well with the activities observed in the uracil selections (FIG. 3).

The selection system of the invention is capable of identifying an individual KDAC variant with the desired activity in a library of more than three million mutants in a single round. Enzymes could be identified to remove typical protection groups for lysine side chains active enough to activate a sufficient amount of Ura3 enzyme to sustain cell growth in the absence of uracil. The selection system can be easily modified to select other KDAC mutant libraries and other lysine modifications. It may also be used to design selective mutant/inhibitor pairs by a bump-and-hole strategy. Enzymes catalysing bioorthogonal reactions are the key to success for the development of safe prodrug strategies in cancer therapy. Presently, enzymes to activate prodrugs are either of human origin (with the disadvantage of being present in other tissues and therefore causing side effects) or from a different organism (with the disadvantage of being immunogenic). KDAC variants of the invention with bioorthogonal activity evolved from a parent enzyme of human origin combine the advantages of both approaches.

TABLE 1

| | | Growth on -ura | | | | | DLR activity [rel. to wild-type] | | | |
|---|---|---|---|---|---|---|---|---|---|---|
| Name | Mutations | AcK | CrK | BocK | AlocK | PrK | AcK | CrK | BocK | AlocK |
| Dealocase-1 | A37S Y53W R56W I92V | n.d. | + | + | + | − | 0.49 | 5.73 | 2.88 | 10.83 |
| SEQ ID 2 | | | | | | | | | | |
| Dealocase-2 | A37G R56G I92V | n.d. | + | + | − | − | 1.19 | 2.11 | 0.77 | 10.23 |
| SEQ ID 3 | | | | | | | | | | |
| Dealocase-3 | I131S V148L | − | + | − | + | n.d. | 0.35 | 1.00 | 0.65 | 6.00 |
| SEQ ID 4 | | | | | | | | | | |
| Debocylase-1 | I131V V148L | + | + | + | + | + | 1.25 | 2.90 | 8.65 | 8.35 |
| SEQ ID 5 | | | | | | | | | | |
| Decrotonylase-1 | A37R Y53G R56T I92R V148L | − | + | − | + | n.d. | 0.00 | 0.10 | 0.04 | 0.02 |
| SEQ ID 6 | | | | | | | | | | |

### Example 6—Development of Humanized Variants

Humanized deacetlyases, i.e. mutant polypeptide of the invention, have been developed. The advantage of a human origin is that there will be no, or only a very reduced, immune reaction in the human organism. For this purpose, the enzymes SirT1, SirT2 and SirT3 are cloned in a manner analogous to the cloning of E. coli cobB. Cloned enzymes are characterized for their ability to activate the marker protein Ura3 K93ac by demodifying the essential lysine residue. Subsequently, mutant libraries are built based on the active variant enzymes. This process is identical to the above-described process based on E. coli cobB.

Inactive precursor molecules of toxic substances are used in cancer therapy, as it is part of the present invention. For this purpose, toxic peptides are modified at their essential lysine residues using protection groups, acetylation and the like. The resulting peptides are tested on human cell lines for toxicity, whereby a low toxicity is preferred. The evolved deacetylases are then characterized for their ability to remove the protection groups and to activate the pro-toxin.

The evolved human deacetylases are tested in human cancer cell lines. For this purpose, the polypeptides are expressed in those cell lines. Subsequently, the cell lines are administered with the pro-toxin peptides to test the ability of the deacetylases to activate them and to provide its effects on the cancer cell line.

### Example 7—KDAC Assay Using Firefly Luciferase K529ac

Materials

Plasmids

pCDF-PyIT-FLuc(opt)His₆-K529TAG: The gene for Firefly Luciferase codon-optimized for expression in E. coli and containing an amber codon replacing the codon for Lys-529 as well as a C-terminal His₆-Tag was custom synthesized by Genscript and cloned into NcoI/XhoI of pCDF-PyIT (Neumann et al., Nat Chem Biol 4 (2008) 232-234). pBK-AcKRS3opt (expressing acetyl-lysyl-tRNA synthetase with mutations improving tRNA binding) was generated from pBK-AcKRS3 by three rounds of QuickChange mutagenesis introducing mutations V31I, T56P, H62Y and A100E (Neumann et al., Molecular Cell 36 (2009) 153-163).

pBK-His₆-CobB: A PCR product encoding His₆-CobB under the control of an arabinose inducible promoter was amplified from CobB subcloned in a pBAD plasmid. The DNA fragment was digested with BglII/StuI and cloned into BamHI/StuI of pBK-PyIS (Neumann et al., Molecular Cell 36 (2009) 153-163).

pBK-His₆-hsHDAC8: His₆-hsHDAC8 gene was custom synthesized by GeneArt, amplified introducing NcoI/XbaI sites and cloned into NcoI/XbaI of pBK-His₆-CobB (replacing His₆-CobB).

pBK-His₆-TEV-hsSirT2 and pBK-His₆-TEV-hsSirT3: The catalytic domain of SirT2 (56-356) and SirT3 (118-399) was amplified from pGEX-TSS-TEV-SirT2/3 introducing NcoI/XbaI sites, His₆-tag and TEV site and cloned into pBK-His₆-hsHDAC8 using the NcoI and XbaI sites. A frameshift in SirT3 was removed by QC.

Expression of KDACs

E. coli BL21 DE3 RIL was transformed with the respective pBK plasmids for CobB, HDAC8, SirT2 or SirT3. Cells were incubated at 37° C. in 10 mL LB medium (50 μg/mL kanamycin) overnight, used to inoculate 1 L LB medium (50 μg/mL kanamycin) and grown to an OD₆₀₀ of 0.3. The temperature was reduced to 30° C. for 1 h before expression was induced by addition of arabinose to a final concentration of 0.2%. Cells were harvested after 16 h by centrifugation (20 min, 6000 rpm, 4° C.). The cell pellets were washed with PBS and stored at −20° C.

Purification of KDACs

Cell pellets were thawed on ice and resuspended in HEPES-Ni-NTA wash buffer (20 mM HEPES, 200 mM NaCl, 20 mM imidazole, 1 mM DTT; pH 7.5 [CobB/HDAC8] or 8.0 [SirT2/3]) supplemented with lysozyme (˜0.5 mg/mL), DNase (1 mg) and protease inhibitors (1 mM PMSF and 0.5× Roche Protease Inhibitor cocktail). Lysis was preformed using a pneumatic cell disintegrator. The cell debris was removed by centrifugation (20 min, 20,000 rpm, 4° C.) and HisPur™ Ni²⁺-NTA Resin (2 mL in 50 mL Solution) was added to the supernatant. After 1 h at 4° C. the suspension was loaded on a plastic column (BioRad, München) with a frit and washed with HEPES-Ni-NTA wash buffer. Protein was eluted in 4 mL Ni-NTA wash buffer containing 200 mM imidazole. The eluate was concentrated and the buffer was exchanged to gelfiltration buffer before loading on a HILoad™ 26/70 Superdex™ 200 size-exclusion chromatography column (GE healthcare, UK) preequilibrated with gel filtration buffer (20 mM HEPES, 100 mM NaCl, 10 mM DTT, pH 7.5 [CobB/HDAC8] or 20 mM Tris/HCl, 50 mM NaCl, pH 8 [SirT2/3]). Absorption at 280 nm was monitored and 5 mL fractions collected. Fractions containing protein were analyzed by SDS-PAGE, pooled and concentrated in a microfiltrator (Amicon Ultra-15 Centrifugal Unit, 10 kDa, Merck Millipore). The protein was aliquoted (50 μL), flash frozen in liquid nitrogen and stored at −80° C.

Purification of Firefly Luciferase K529ac

E. coli BL21 DE3 were transformed with plasmids pCDF-PyIT-FLuc(opt)His6-K529TAG and pBK-AcKRS3opt. Cells were grown in LB medium in the presence of antibiotics (50 μg/μl spectinomycin and 50 μg/μl kanamycin) to maintain the plasmids, 5 mM acetyl-lysine and 20 mM nicotinamide at 37° C. to an OD600 of 1.0. Then, cells were shifted to 30° C. and protein expression induced by the addition of 1 mM IPTG. After further 4 h at 30° C. cells were harvested by centrifugation, washed with PBS and lysed in Ni-wash buffer (20 mM Tris/HCl, 10 mM imidazole, 200 mM NaCl, 10 mM DTT, 2 mM PMSF, 0.5× Roche Protease Inhibitor cocktail, pH 8) containing 20 mM nicotinamide by addition of lysozyme. The sample was sonicated for 2 min (Power output level 5, duty cycle 50%) and centrifuged (20 min, 50,000 g, 4° C.). The supernatant was supplemented with 500 μl Ni-NTA-beads. After two hours incubation with agitation at 4° C. beads were washed with 30 ml Ni-wash buffer and bound proteins eluted in Ni-wash buffer supplemented with 200 mM imidazole. The eluate was used without modification as deacetylase substrate.

Luciferase-Based KDAC Assay

Typical endpoint deacetylation reactions contain: 30 nM Firefly Luciferase K529ac, 1 mM NAD⁺, 1 μg/ml KDAC in 50 μl KDAC buffer (25 mM Tris/HCl pH 8.0, 137 mM NaCl, 2.7 mM KCl, 1 mM MgCl₂, 1 mM DTT, 1 mg/ml BSA). The reactions are incubated for 1 h at 25° C. Luciferase activity is then assayed by addition of an equal volume of a mixture containing 40 mM Tricine, 200 μM EDTA, 7.4 mM MgSO₄, 2 mM NaHCO₃, 34 mM DTT, 0.5 mM ATP and 0.5 mM luciferin, pH 7.8⁴. Luminescence is quantified using a FluoStar Omega Microplate Reader (BMG Labtech).

The continuous FLuc-based KDAC assay was set up by mixing all the components of the endpoint assay immediately. Usually NAD⁺ was omitted initially and added from a 20-fold stock solution after 5 min preincubation to start the reaction. Luminescence was recorded every minute over a period of 30 min. KDAC activity was calculated from the slope of the linear phase of the reaction.

Fluor-De-Lys KDAC Assay

Typical deacetylation reactions were identical to Luciferase-based assays but containing 10 μg/ml KDAC and 10 μM Fluor-de-Lys peptide (Ac-Gly-Gly-Lys(ac)-AMC). Conditions were derived from Zhou et al., Molecules 22 (2017) 1348). After incubation for 1 h at 25° C. trypsin and 120 mM nicotinamide were added to the reaction and the reactions were further incubated for 15 min at 37° C. Coumarin fluorescence (ex. 355 nm, em. 460 nm) was then measured using a FluoStar Omega Microplate Reader (BMG Labtech).

Results

It was tested whether purified FLuc K529ac can be used to quantify KDAC activity by incubating it with various different KDACs (FIG. 7). Prior to treatment with a KDAC the enzyme produced very little bioluminescence. After incubation with various KDACs the luminescence increased up to 130 fold. Hence, FLuc K529ac is a substrate for KDACs and a highly sensitive reporter enzyme for KDAC activity.

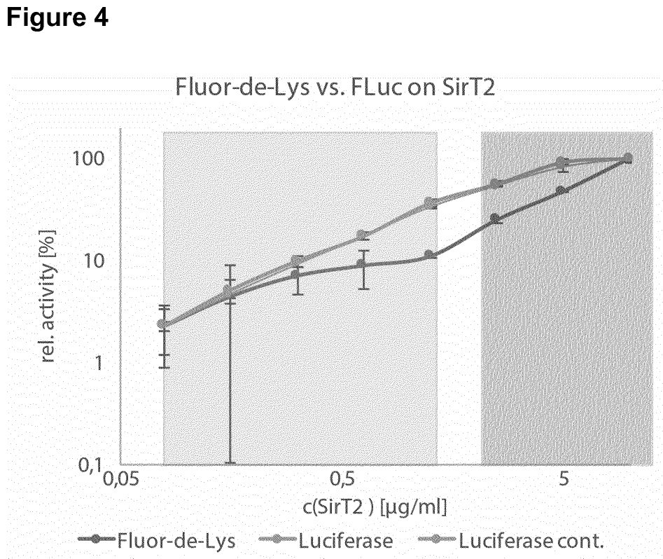

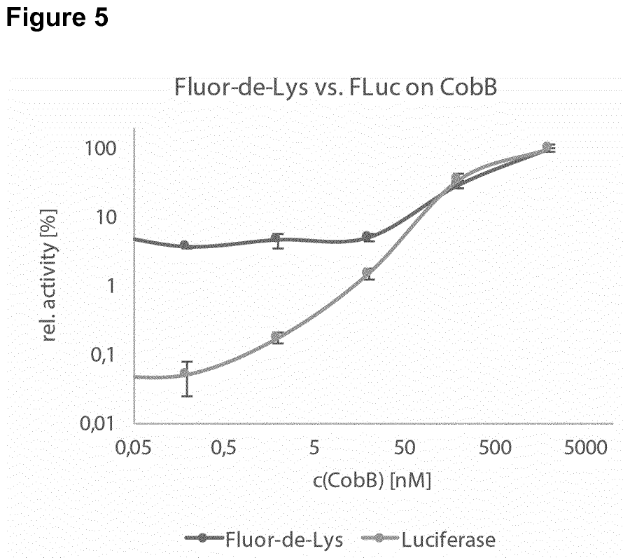

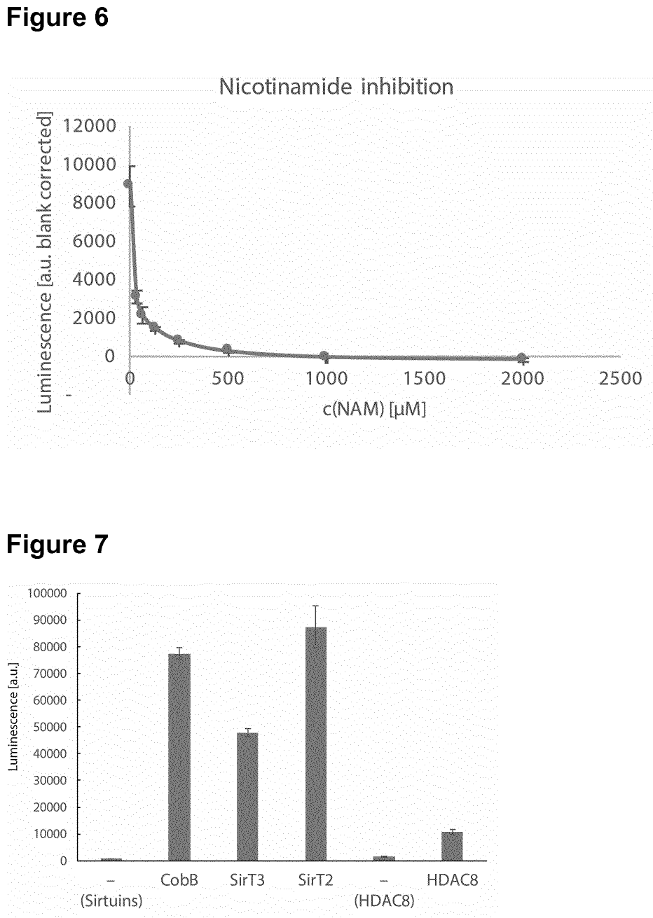

The assay shows a linear response to increasing KDAC concentrations over a range of 2-3 orders of magnitude (FIGS. 4 & 5). Addition of nicotinamide to the deacetylation reaction of SirT2 inhibited the assay with an IC₅₀ of 30 μM (FIG. 6).

### Example 8—KDAC Inhibitor Screening Method

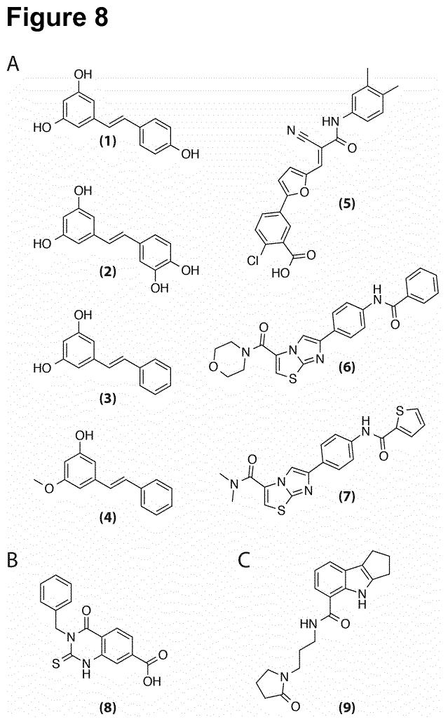

It was tested whether the FLuc-based KDAC assay of the invention is suitable for screening KDAC inhibitors. Therefore, a set of 351 compounds was composed with similarity to known sirtuin inhibitors. The effect of the compounds was analyzed at 10 μM on SirT2 activity using the FLuc K529ac assay in endpoint format. The initial screen identified eight compounds inhibiting the assay >50% and one activating more than 1.5 fold (FIG. 8). Compound 9 showed direct inhibition of FLuc, while the remaining seven inhibitors displayed IC₅₀ values against SirT2 of 3-15 μM. The compounds with the highest potency are resveratrol (1) and piceatannol (2). Piceatannol had previously been shown to inhibit SirT2. The structurally very similar resveratrol is a known activator of yeast Sir2 and SirT1. The effect on other sirtuins strongly depends on the combination of KDAC and substrate and can be either activating or inhibitory. In sum, a highly reliable KDAC assay is presented with exceptional sensitivity. The assay is convenient and fast and can be performed in a continuous format. By producing the accordingly modified FLuc, it would be straightforward to adapt the assay to measure the removal of crotonyl, butyryl, propionyl or 2-hydroxyisobutyryl groups from lysine residues. Seven compounds were identified, which inhibit FLuc K529ac deacetylation by SirT2 at low micromolar concentrations. Compound 5 is structurally similar to AGK-2, which inhibits SirT2 with an IC₅₀ of 3.5 μM. Compounds 6 and 7 are similar to SRT1720, which was initially reported as a potent activator of SirT1 and later shown to specifically enhance its interaction with fluorophore-labeled peptides. Piceatannol (2) and particularly resveratrol (1) are familiar sirtuin activators. It is therefore surprising that resveratrol and structurally similar compounds are the most active inhibitors of SirT2 identified in the screen. However, resveratrol specifically activates fluorophore-labeled peptide substrates by stabilizing the enzyme-substrate complex. Resveratrol's effect on sirtuin activity is highly dependent on the sirtuin-substrate combination and has indeed been shown to be inhibitory for SirT3 by enforcing an unproductive conformation of the enzyme-substrate complex. The 2-mercapto-quinazoline derivative 8 is structurally similar to thiobarbiturates, which have been reported to inhibit SirT2 at low micromolar concentration. Thus, the herein provided assay provides a reliable tool for screening of chemical compounds for their ability to inhibit KDAC activity.

1. A method of selecting a polypeptide having lysine demodification, in particular lysine deacylation, activity from a collection of polypeptides, wherein the method comprises the following steps:
(a) incubating said polypeptide with a peptide or polypeptide comprising an essential lysine residue inactivated by a modification, in particular an acylation, of said essential lysine residue; and
(b) selecting said polypeptide based on the ability of said polypeptide to activate said peptide or polypeptide comprising the inactivated essential lysine residue,
wherein said polypeptide and said peptide or polypeptide comprising an inactivated essential lysine residue are incubated in a biological cell.

2. The method of claim 1 further comprising the following counter-selection steps:
(c) incubating a polypeptide selected in step (b) with a peptide or polypeptide comprising an essential lysine residue differentially inactivated by a modification different from the modification used in step (a); and
(d) selecting said polypeptide based on the inability of said polypeptide to activate said peptide or polypeptide comprising said differentially inactivated essential lysine residue.

3. A method of screening a diverse collection of polypeptides for a polypeptide having lysine demodification, in particular lysine deacylation, activity, wherein the method comprises the following steps:
(a) incubating said diverse collection of polypeptides with a luciferase comprising an inactivated residue K529, wherein said residue is inactivated by a modification, in particular an acylation; and
(b) selecting said polypeptide based on the ability of said polypeptide to activate said luciferase,
wherein said diverse collection and said luciferase are incubated in a diverse collection of biological cells; particularly wherein said luciferase is Firefly luciferase according to SEQ ID NO: 7.

4. The method of claim 3 further comprising the following counter-screening steps:
(c) incubating a polypeptide selected in step (b) with a luciferase comprising an inactivated residue K529, where said residue is differentially inactivated by a modification different from the modification used in step (a); and
(d) screening said polypeptide based on the inability of said polypeptide to activate said luciferase comprising said differentially inactivated residue K529.

5. A method of screening or selecting a KDAC inhibitor from a diverse collection of putative KDAC inhibitors, wherein the method comprises the following steps:
(a) incubating a polypeptide having a lysine demodification, in particular a lysine deacylation, activity with a member of said diverse collection;
(b) adding a peptide or polypeptide comprising an essential lysine residue inactivated by a modification, in particular an acylation, of said essential lysine residue; and
(c) identifying a KDAC inhibitor by the ability to inhibit the demodification, in particular the deacetylation, activity of said polypeptide,
wherein the KDAC inhibiting activity of said KDAC inhibitor is reciprocal to the activity of said polypeptide to activate the peptide or polypeptide comprising the inactivated essential lysine residue; in particular, wherein the method is performed in a biological cell.

6. The method of claim 1, 2, or 5, wherein the peptide or polypeptide comprising an essential lysine residue inactivated by a modification is OMP decarboxylase.

7. The method of claim 6, wherein OMP decarboxylase is buddying yeast OMP decarboxylase (Ura3) or E. coli pyrF.

8. The method of claim 7, wherein OMP decarboxylase is buddying yeast OMP decarboxylase (Ura3) comprising an inactivated residue K93.

9. The method of claim 5, wherein the peptide or polypeptide an essential lysine residue inactivated by a modification is a luciferase comprising an inactivated residue K529; particularly wherein said luciferase is Firefly luciferase according to SEQ ID NO: 7.

10. The method of claim 9, wherein the luciferase comprises an amino acid sequence having at least 90% sequence homology to SEQ ID NO: 7, particularly wherein said luciferase is Firefly luciferase comprising the sequence according to SEQ ID NO: 7.

11. The method of any one of claims 1 to 10, wherein the essential lysine residue is inactivated by acylation or an alternative protection group, particularly by acylation.

12. The method of claim 11, wherein the essential lysine residue is inactivated by acylation with an acyl group selected from the groups of acetyl, crotonyl, tert.-butyloxycarbonyl (Boc), allyloxycarbonyl (Aloc), propargyloxycarbonyl (Poc), benzyloxycarbonyl (Z), 2,2,2-trichloroethyloxycarbonyl (Troc), azidomethoxycarbonyl (Azoc), 2-chlorobenzyloxycarbonyl (Cl—Z) and trifluoroacetyl (tfa).

13. The method of any one of claims 1 to 12, wherein the biological cell is a bacterial cell, in particular wherein the bacterial cell is an E. coli cell.

14. The method of claim 13, wherein the bacterial cell is an E. coli cell, which lacks a gene encoding pyrF and/or cobB and/or wherein the activity of pyrF and/or cobB is inhibited in said E. coli cell.

15. A luciferase, in particular a luciferase comprising an amino acid sequence having at least 90% sequence homology to SEQ ID NO: 7, wherein the polypeptide comprises an inactivated lysine residue at a position corresponding to position 529 of SEQ ID NO: 7; particularly wherein the polypeptide comprises the sequence according to SEQ ID NO: 7.

16. The polypeptide of claim 15, wherein the lysine residue is inactivated by acylation, in particular by acylation with an acyl group selected from the groups of acetyl, crotonyl, tert.-butyloxycarbonyl (Boc), allyloxycarbonyl (Aloc), propargyloxycarbonyl (Poc), benzyloxycarbonyl (Z), 2,2,2-trichloroethyloxycarbonyl (Troc), azidomethoxycarbonyl (Azoc), 2-chlorobenzyloxycarbonyl (Cl—Z) and trifluoroacetyl (tfa).

17. The polypeptide of claim 15 or 16, additionally comprising a purification tag, preferably a 6× His-tag.

18. A nucleic acid encoding the polypeptide of claim 15 or 16, wherein the codon encoding the essential lysine residue is replaced by an amber stop codon.

19. The nucleic acid of claim 18 comprising a nucleic acid sequence having at least 80% sequence homology to SEQ ID NO: 8; particularly a nucleic acid sequence encoding the protein according to SEQ ID NO: 7. wherein the codon encoding the essential lysine residue is replaced by an amber stop codon.

20. A mutant polypeptide comprising an amino acid sequence having at least 99% sequence homology with SEQ ID NOs: 2, 3, 4, 5 or 6 and having lysine demodification, in particular lysine deacylation, activity, wherein the mutant polypeptide is not identical to SEQ ID NO: 1.
